(19) United States
(12) Patent Application Publication
(10) Pub. No.: US 2023/0270199 A1
(43) Pub. Date: Aug. 31, 2023

(54) THERAPEUTIC SHOES AND COMPONENTS FOR USE WITH THERAPEUTIC SHOES

(71) Applicant: Matthew J. Jacobs, Grand Haven, MI (US)

(72) Inventor: Matthew J. Jacobs, Grand Haven, MI (US)

(21) Appl. No.: 18/008,164

(22) PCT Filed: Dec. 13, 2020
(86) PCT No.: PCT/US2020/064734

§ 371 Date: Dec. 3, 2022

Related U.S. Application Data
(60) Provisional application No. 62/947,157, filed on Dec. 12, 2019.

(51) Int. Cl. A43B 3/44; A43B 1/00; A43B 7/14

(52) U.S. Cl. CPC A43B 3/44; A43B 1/0054; A43B 7/14

(57) ABSTRACT

Stability footwear (e.g., a pair of therapeutic shoes) may include at least one stabilizing member, stabilizer, and/or outrigger on a lateral (outer or inner) side of a shoe, substantially adjacent to a sole of a shoe. A stabilizer may be positioned proximate a surface on which the sole rests and extending anywhere from a front, substantially a toe, to a heel of a shoe. The stabilizing member, stabilizer, and/or outrigger may reduce undesired foot roll and consequential ankle injuries and/or falls, by providing additional lateral support that does not compromise mobility or performance of the user.

### TECHNICAL FIELD

The present disclosure generally relates to therapeutic shoes. More particularly, the present disclosure relates to reconfigurable therapeutic shoes, components for use with configuring a pair of therapeutic shoes, and systems and methods for configuring a pair of therapeutic shoes.

### BACKGROUND

Therapeutic shoes may generally improve stability of an individual wearing the shoe. Therapeutic shoes may improve an ability of the individual to walk without falling.

Many individuals may experience improved stability with a proper and consistent foot orientation. Many articles and books have been written which indicate a proper form for a therapeutic shoe and, therefore, most individuals could walk better if they could adopt the suggestions and develop consistency in their foot orientation and stability.

One factor preventing widespread development of proper walking techniques concerns the inability of most individuals to find the time and facilities for therapy. Thus, it is another recognized fact that proper therapeutic shoes, and consistent use thereof, is heretofore developed if long hours of therapy are undertaken.

There are also certain physical factors which are unique to individual therapeutics which prevent improvement in walking. Accordingly, it is desirable to most people to acquire aids which would tend to overcome physical inadequacies, and which would tend to improve their stability.

It is a general object of this disclosure to provide a means adapted to be used in combination with therapeutic shoes which will tend to improve stability and walking of an individual employing such means.

It is a more particular object of this disclosure to provide a means for configuration of a therapeutic shoe which will orient an individual into a more proper pattern to thereby increase the likelihood of stability and walking in a manner desired.

It is a further particular object of this disclosure to provide a means to configure a therapeutic shoe which will facilitate acquisition of stability and a proper walk to, therefore, decrease the necessity for long hours of therapy and to also tend to overcome physical inadequacies of certain individuals.

### SUMMARY

A pair of therapeutic shoes may include a left shoe having at least one left shoe sensor and at least one left shoe stabilizer member. The right shoe may have at least one right shoe sensor and at least one right shoe stabilizer member. At least one left shoe stabilizing member may be dynamically configured based on an output from at least one right shoe sensor and/or at least one right shoe stabilizing member may be dynamically configured based on an output of at least one left shoe sensor.

In another embodiment, a computer-readable medium may store computer-readable instructions that, when executed by a processor, may cause the processor to generate a configuration for at least one therapeutic shoe. The computer-readable medium may include a pedograph data receiving module that, when executed by the processor, may cause the processor to receive pedograph data. The pedograph data may be representative of a distribution of pressure resulting from at least one foot of an individual on a surface. The computer-readable medium may also include a therapeutic shoe configuration data generation module that, when executed by the processor, may cause the processor to generate therapeutic shoe configuration data based on the pedograph data.

In a further embodiment, a method of configuring a therapeutic shoe may include receiving pedograph data that may be representative of at least one of: a footprint of a therapeutic shoe wearer, a previously determined configuration of a therapeutic shoe, or personal data associated with a therapeutic shoe wearer. The method may also include generating therapeutic shoe configuration data based on the pedograph data. The method may further include configuring a therapeutic shoe based on the therapeutic shoe configuration data.

### BRIEF DESCRIPTION OF THE DRAWINGS

These and other objects of the claimed invention will appear hereinafter and for purposes of illustration but not of limitation, specific embodiments of this invention are shown in the accompanying drawings in which:

FIG. 1A depicts a perspective view of an example pair of therapeutic shoes;

FIG. 1B depicts a top plan view of an example pair of therapeutic shoes;

FIG. 10 depicts a side profile view of an example pair of therapeutic shoes;

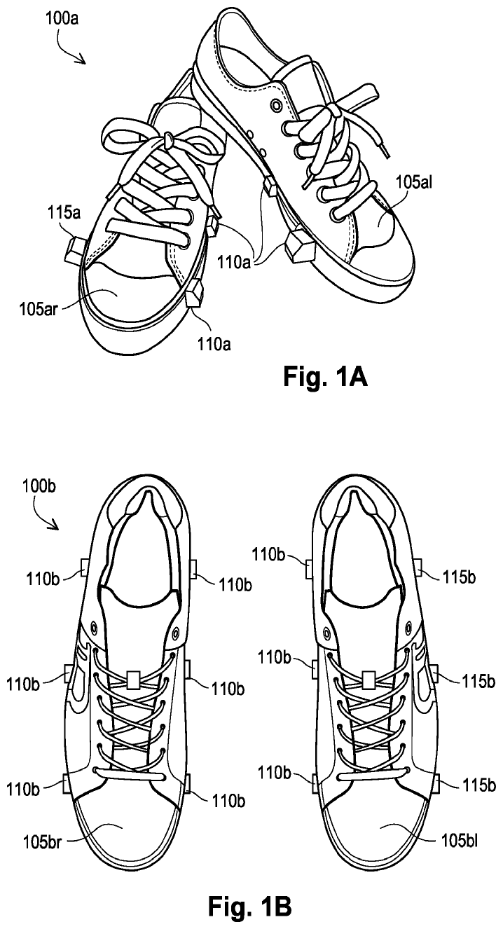

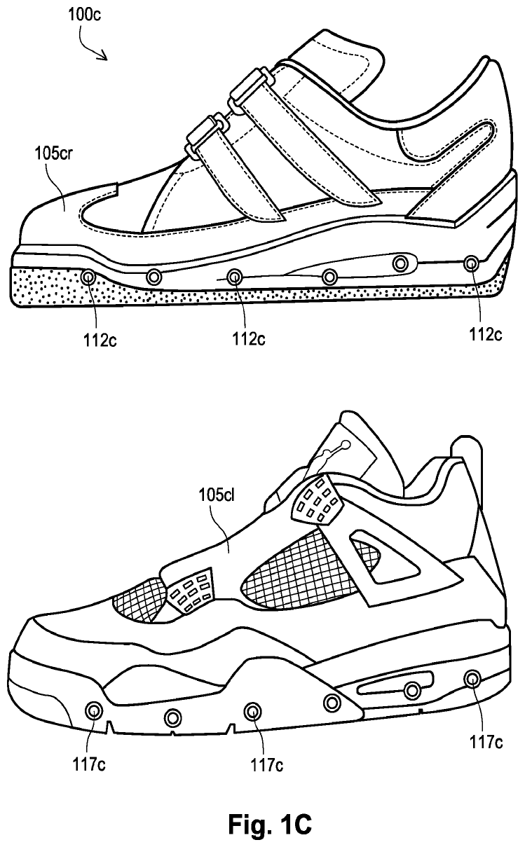

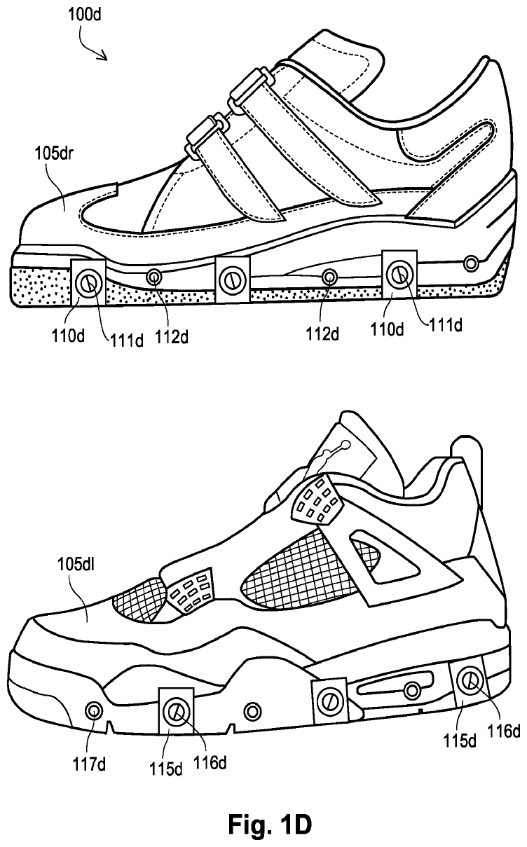

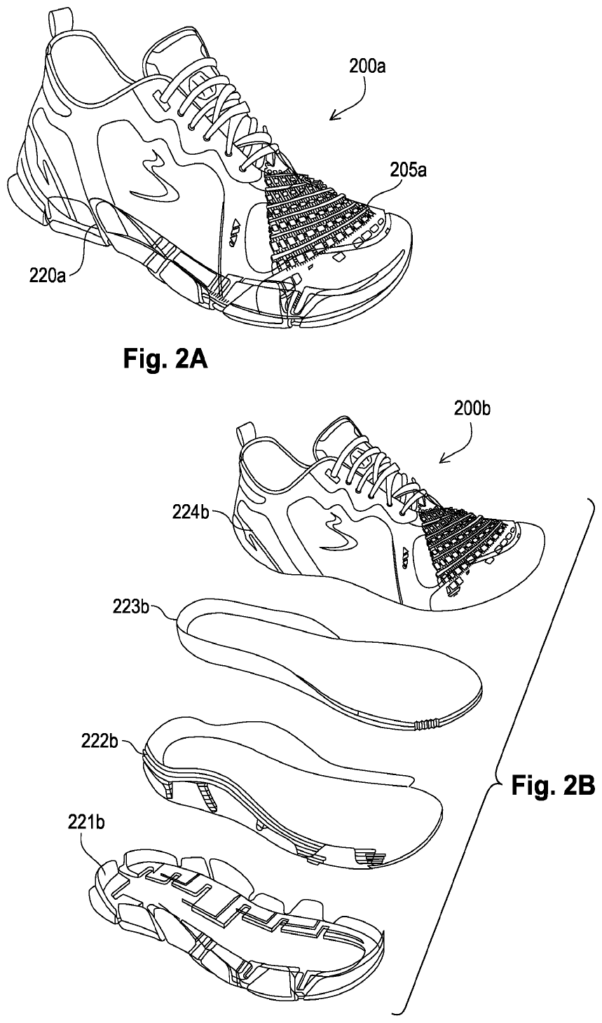

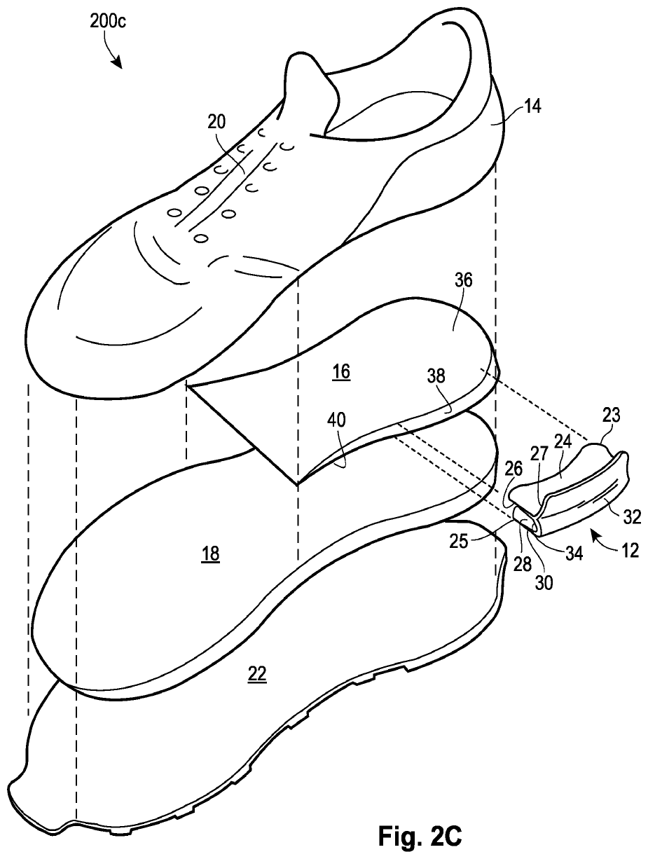

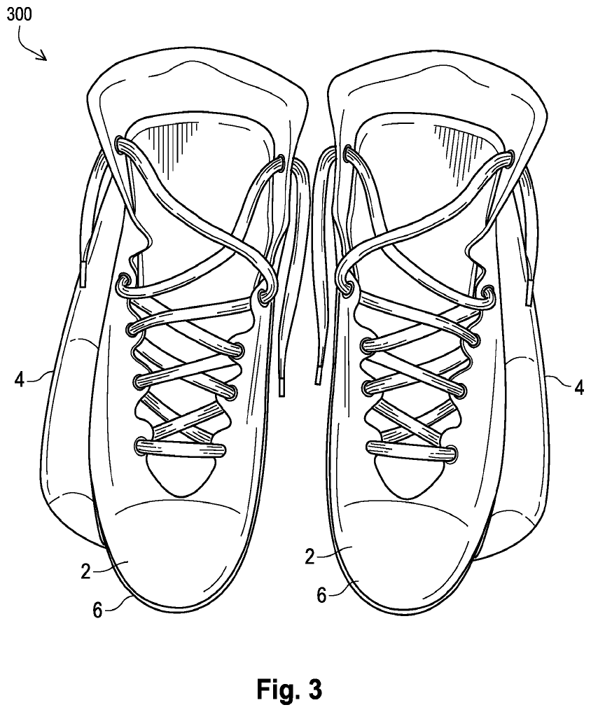

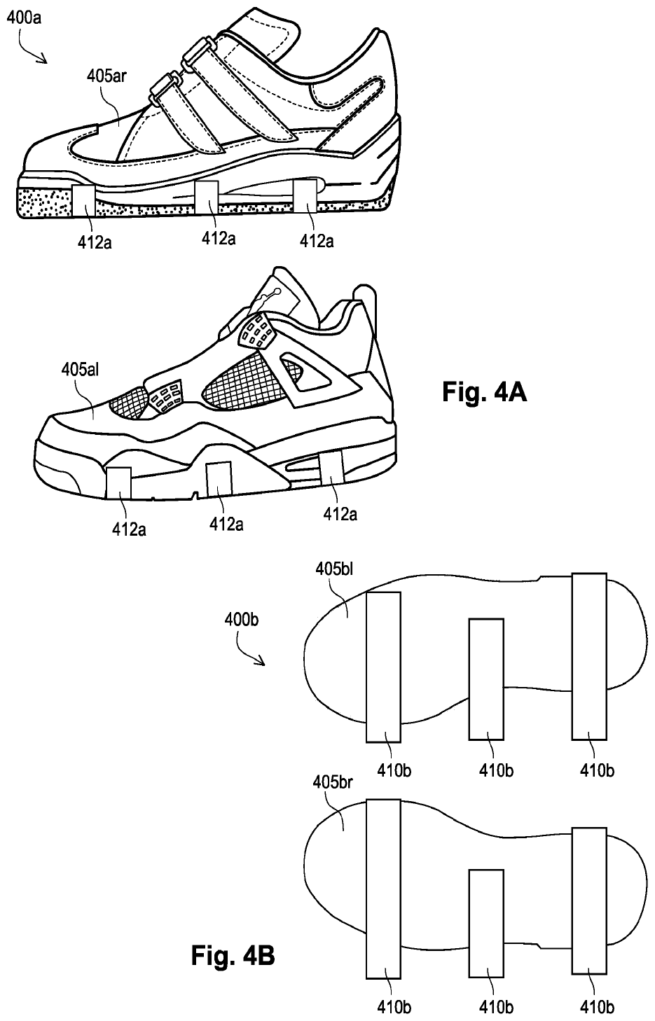

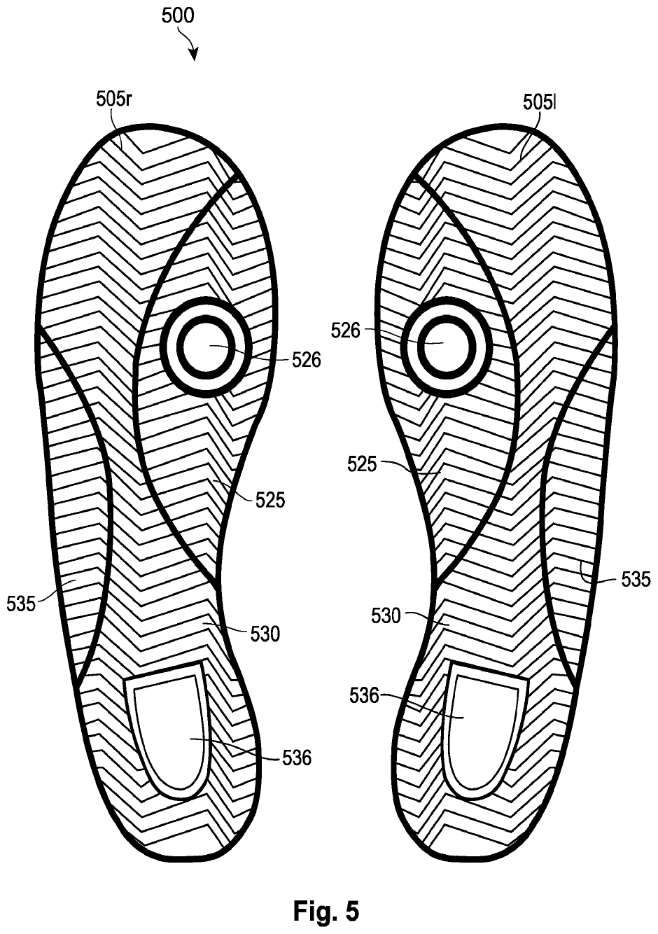

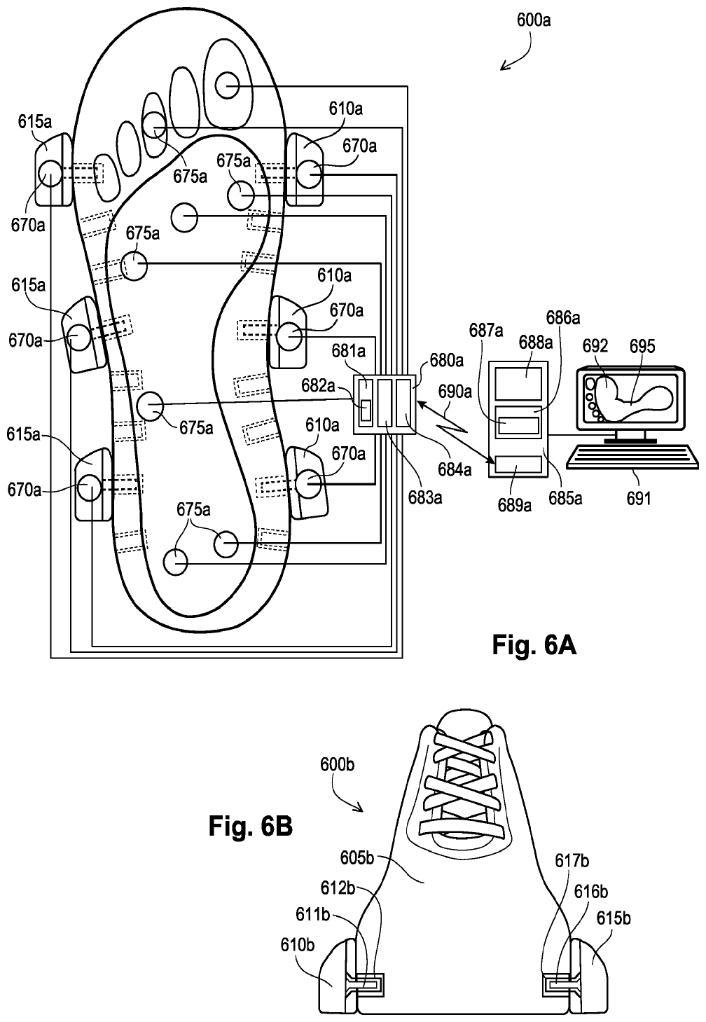

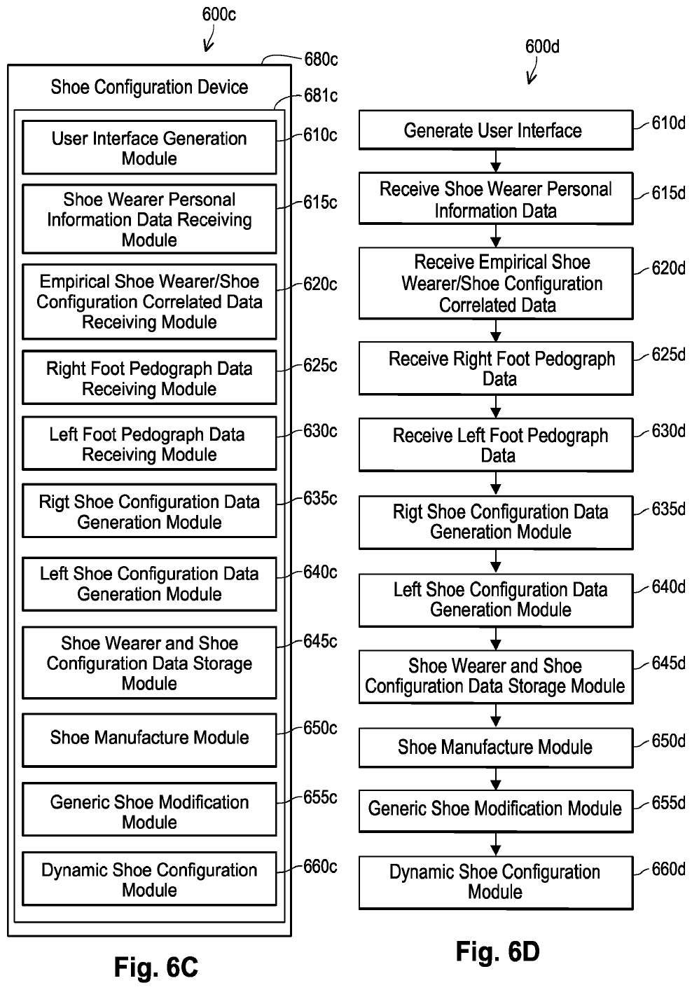

FIG. 1D depicts a side profile view of an example pair of therapeutic shoes;

FIG. 2A depicts a perspective view of an example therapeutic shoe;

FIG. 2B depicts an exploded perspective view of an example pair of therapeutic shoes;

FIG. 2C depicts an exploded perspective view of an example pair of therapeutic shoes;

FIG. 3 depicts a top plan view of an example pair of therapeutic shoes;

FIG. 4A depicts a side profile view of an example pair of therapeutic shoes;

FIG. 4B depicts a bottom plan view of an example pair of therapeutic shoes;

FIG. 5 depicts a bottom plan view of an example pair of therapeutic shoes; and

FIGS. 6A-D depict an example system for configuring a pair of therapeutic shoes.

### DETAIL DESCRIPTION

Therapeutic shoes are provided that may include a left shoe and/or a right shoe having at least one laterally extending stabilizer member (e.g., an outrigger). A therapeutic shoe may be configured to provide a level stability (e.g., level 1 stability, level 2 stability, level 3 stability, etc.) for a particular shoe wearer. Stability level 1 may be, for example, associated with providing stability with no removable stabilizing members attached to an associated therapeutic shoe. Stability level 2 may be, for example, associated with providing stability with at least one relatively short removable stabilizing members (e.g., at least one 0.25 inches to 1.0 inch stabilizing member) attached to an associated therapeutic shoe. Stability level 3 may be, for example, associated with providing stability with at least one relatively long removable stabilizing member (e.g., at least one 1.0 inch to 2.0 inches stabilizing member) attached to an associated therapeutic shoe.

A therapeutic shoe configured with level on stability, for example, may be delivered to a respective shoe wearer along with a plurality of stabilizing members. The level one stability therapeutic shoe may be modified (e.g., by the shoe wearer, by a podiatrist, etc.) using at least one stabilizing member to result in a therapeutic shoe configured with level two or level three stability.

Alternatively, or additionally, a pair of therapeutic shoes may include at least one shoe having at least one sensor and at least one stabilizing member having at least one actuator. The at least one therapeutic shoe may include a stability controller configured to dynamically adjust at least one stabilizing member via, for example, the at least one actuator based on an output from the at least one sensor.

Any given therapeutic shoe may be configured, at least in part, based on input provided by a pedorthist and/or a podiatrist. For example, a pedograph may be generated for a particular shoe wearer. The systems and methods of the present disclosure may perform analysis of a footprint generated by a device used to obtain footprints for analysis. Similarly, an instrument to record and study a gait of a shoe wearer.

Turning to FIG. 1A, a pair of therapeutic shoes 100a may include a right foot shoe 105ar having at least two stabilizing members 110a extending laterally from an inner surface and at least one stabilizing member 115a extending laterally from an outer surface. The pair of therapeutic shoes 100a may include a left foot shoe 105a1 having at least two stabilizing members 110a extending laterally from an inner surface.

With reference to FIG. 1B, a pair of therapeutic shoes 100b may include a right foot shoe 105br having at least three stabilizing members 110b extending laterally from an inner surface and at least three stabilizing members 115b extending laterally from an outer surface. The pair of therapeutic shoes 100b may include a left foot shoe 105b1 having at least three stabilizing members 110b extending laterally from an inner surface and at least three stabilizing members 115b extending laterally from an outer surface.

Turning to FIG. 1C, a pair of therapeutic shoes 100c may include a right foot shoe 105cr having a plurality of stabilizing member attachments 112c (e.g., threaded receptacles, friction fit receptacles, interference fit receptacles, snap in receptacles, magnetic device, etc.) along an inner surface and a plurality of stabilizing member attachments 117c (e.g., threaded receptacles, friction fit receptacles, interference fit receptacles, snap in receptacles, magnetic device, etc.) along an outer surface. While the therapeutic shoe 105cr may be illustrated as a right foot shoe 105cr, the therapeutic shoe may be a similar left foot shoe. While the therapeutic shoe 105cr may be illustrated as having six stabilizing member attachments 112c and six stabilizing member attachments 117c, any given therapeutic shoe 105cr may include fewer (e.g., three stabilizing member attachments 112c and/or three stabilizing member attachments 117c) or more stabilizing member attachments 112c and/or stabilizing member attachments 117c.

With reference to FIG. 1D, a pair of therapeutic shoes 100d may include a right foot shoe 105dr having a plurality of stabilizing members 110d removable attached to stabilizing member attachments 112d, via respective fasteners 111d (e.g., screws, pins, snaps, magnets, pressure sensitive adhesive, etc.) along an inner surface, and a plurality of stabilizing members 115d removably attached to stabilizing member attachments 117d, via respective fasteners 116d (e.g., screws, pins, snaps, magnets, pressure sensitive adhesive, etc.) along an inner surface along an outer surface. While the therapeutic shoe 105dr may be illustrated as a right foot shoe 105dr, the therapeutic shoe may be a similar left foot shoe. While the therapeutic shoe 105dr may be illustrated as having three stabilizing members 115d and three stabilizing members 110d, any given therapeutic shoe 105dr may include fewer (e.g., one stabilizing member 115d and/or two stabilizing members 110d) or more stabilizing members 115d and/or stabilizing members 110d.

A therapeutic shoe in accordance with the present disclosure may be particularly designed for association with any conventional therapeutic shoe 100a-d, 10. A stabilization means may comprise at least one support which is to be associated adjacent to an inner or outer edge of a therapeutic shoe 100a-d, 10. Accordingly, the support is associated with the therapeutic shoe 100a-d, 10 relative to the intended direction of movement of the wearer of the shoe 100a-d, 10.

The support means may include an integral member which may, for example, include a triangularly shaped portion and which may define a ground engaging face and a shoe 100a-d, 10 engaging face. The cross section of the member may be such that a thicker portion of the triangle will be located near the edge of the shoe 100a-d, 10 while a thinner portion may be located inwardly thereof. Accordingly, the support member may provide for an inward or outward tilt of the shoe 100a-d, 10.

The support may be configured to be removed from the therapeutic shoe 100a-d, 10. On the other hand, a fastener may be provided for holding the support securely in place when a user walks. Accordingly, an individual employing the means of this invention can place a support on a shoe 100a-d, 10 when preparing to walk and can then quickly remove the support while performing other tasks. If the device is used for improving a person's stability, then there may be no need to remove the support.

FIGS. 1A-D illustrate a therapeutic shoe 100a-d which may include a sole portion formed thereon or fitted thereto. The support member 110a-d, 115a-d of this invention is fitted in place on a shoe 100a-d.

The support member illustrated in FIGS. 1A-D may, for example, comprise a lateral portion and two inwardly extending portions having a triangular cross section. These portions may define ground engaging and shoe 100a-d engaging faces. Intermediate the portions, there may be provided a pair of prongs which may extend inwardly. These prongs may define an opening between them, and this opening may be adapted to receive a shank of a fastener 111b,d threaded into the sole or socket 112e of the shoe 100a-d. As illustrated, this fastener may be located at an inside or outside location of the shoe 100a-d. The fastener 111b,d may comprise a screw adapted to be threaded into the bore 112e.

Alternatively, a channel shaped member may define an opening for receiving a stabilizing member 110a-d, 115a-d, and in-turned walls of this channel shaped member may define an opening between this member and the outer surface of the wall. The stabilizing members 110a-d, 115a-d may be, for example, adapted to be rotatably or linearly received in this opening whereby the support can easily slide into place.

A base portion of a shoe 100a-d, 10, normally integral with a sole, can serve to define the channel for the stabilizing member 110a-d, 115a-d by turning the edges inwardly to form walls corresponding to the walls.

A stabilizing member 110a-d, 115a-d may include an up-turned edge which may fit around an outer edge of the sole. Inward movement of the support member 16 is thus limited by means of this edge.

Turning to FIG. 2A, a pair of therapeutic shoes 200a may include a therapeutic shoe 205a having an integrally formed sole with stabilizing members 220a. With further reference to FIG. 2B, a pair of therapeutic shoes 200b may include an upper portion 224b. The upper portion 224b may, for example, include a breathable mesh and/or fabric. The upper portion 224b may include a reflective material for safety. The upper portion 224b may, for example, include laces and/or Velcro flaps to secure the therapeutic shoe 205b to a respective foot of a shoe wearer. The upper portion 224b may include a padded lining and/or heel collar. The upper portion 224b may include a toe box that is wider than a remaining portion.

The therapeutic shoe 205b may include an insole 223b. The insole 223b may include, for example, an EVA foam sock liner. The therapeutic shoe 205b may include a midsole 222b. The midsole 222b may include at least one side and/or a heel made of EVA foam that is, for example, heavier density than the EVA foam of the insole 223b. the therapeutic shoe 205b may include a tread 221b. The tread 221b may aid in side-to-side and/or front-to-back grip stability with respect to an associated support surface. The tread 221b may include, for example, “vibram” like material. While not shown in FIG. 2B, the therapeutic shoe 205b may include a heel cup and/or an arch support. The heel cup and/or the arch support may include, for example, a hard plastic.

The therapeutic shoes shown in FIGS. 2A-C and 3 may comprise a support member which is adapted to be more permanently attached to the therapeutic shoe 10. In this case, an opening defined by this member may be adapted to receive a fastener 111a-d which can be screwed into the appropriate bore 112e. This design is particularly suitable for a person who wishes to use the support only while practicing walking. The support can be put on the shoe 100a-d, 10 and maintained thereon during the duration of the practicing and then the stabilizing member 110a-d, 115a-d can be replaced when the therapeutic shoe 100a-d, 10s are to be used.

One advantage of the therapeutic shoes of the present disclosure is an ability to the support a person a more proper and consistent foot orientation. With the support in place, the shoe 100a-d, 10 may be tilted inwardly, and it is believed that this tends to provide a more satisfactory weight distribution when walking. Furthermore, it has been found that a more desirable stride may result when a person employs this support. Thus, it has long been recognized that many individuals tend to sway during a walk rather than to walk in line with a more or less stationary vertical line. By providing the means of this invention, it is uncomfortable to move the hips laterally and accordingly, there may be a greater natural tendency toward proper walking. It will be understood that the shoe 100a-d, 10 may improve a walking path relative to the intended direction of a line of travel of the individual wearing the shoe 100a-d, 10.

The modification illustrated in FIGS. 1A-C, 2A-C and 3 may be considered advantageous primarily for use in therapy since, in such circumstances, walking from approximately the same location and a great deal of walking is not required. However, the more readily removable modification is considered to embody clear inventive features for the reason that this modification can be used on the therapeutic course as well as during a therapy session. The design of the support permits easy removal and replacement so that the user can walk in a comfortable fashion between shots. The use of a fastener such as shown at 34 for the fastening means is also important in this regard since the therapeutic shoe 100a-d, 10 will have a natural feel when the support is not in place.

It will be apparent to those skilled in the art that various other means could be employed for removably attaching the stabilizing member 110a-d, 115a-d to a therapeutic shoe 100a-d, 10 in the position shown. In this connection, a bracket could be fixed to the shoe 100a-d, 10 in the desired position and prongs or other extending means associated with the support could fit into this bracket. Thus, a function similar to that referred to with respect to the modification of FIGS. 1A-D, 2A-C and 3 could be accomplished without the use of a fastener 111a-d, 116a-d. In addition, it is contemplated that an opening for receiving an extension on the stabilizing member 110a-d, 115a-d could be formed in the sole of the shoe 100a-d, 10. Finally, it is contemplated that a support of the nature shown in FIGS. 1A-D, 2A-C and 3 could be formed in whole or in part of magnetized material with the recess in the support being shaped to receive the magnetic spike of a shoe 100a-d, 10 or an independent magnetic means fixed to the shoe 100a-d, 10.

With additional reference to FIG. 2C, a therapeutic shoe 200c may be configured as a running shoe 10. An upper portion 20, which may include heel counter 14, heel wedge 16, midsole 18, 1 outsole 22, and the stabilizer device 12. Stabilizer 12 is attached permanently to the inside of heel wedge 16 by using glue or other means. The bottom surface 26 of upper plate 23, of stabilizer device 12, substantially conforms and is joined to top surface 36, of 2 heel wedge 16, while inside surface 34 of side wall 32 is joined to surface 38 of wedge 16 and surface upper 28 of lower plate 25 is joined to surface 40. Surface 27 of extension piece 29 which conforms to the outer surface of heel counter 14 is also permanently attached to the 2 heel counter. Upper portion 20, heel wedge 16, with stabilizer 12 attached, midsole 18, and outsole 22, and then joined permanently together. An insole (not shown) may be superposed over wedge 16 and midsole 18.

Turning to FIG. 3, outrigger 4 may be attached to the lateral side of shoe 2. Outrigger 4 may extend almost to the extreme front toe 6 of shoe 2 to substantially the rear heal 8 of shoe 2. Shoe 2 may be made such that sole 12 of shoe 2 generally contacts against surface 10 upon which the shoe rests in a flat or co-planar relationship. As also shown in FIG. 3, outrigger 4 (e.g., a stabilizer member) may be positioned on shoe 2 slightly above surface 10. In this position, most or all of outrigger 4 may not contact surface 10 when shoe 2 is sitting flat.

In normal movement of the foot during activities, such as athletic activities, the outrigger ay periodically contact the floor, ground, or other surface that sole 12 of the shoe contacts during use, but would not interfere with the user's activities. A portion of the bottom of outrigger 4 may contact the surface on which the sole rests when the shoe is being worn with the force of the weight and stance of the user determining the distribution of weight and accompanying distortion on shoe 2.

As shown in FIG. 3, outrigger 4 is may extend along the outer edge of the outside of each shoe 2 for substantially the entire length of shoe 2 from toe 6 to heel 8. However, in order to not restrict natural movement, outrigger 4 generally would not extend in front of toe 6 of shoe 2. In contrast, outrigger 4 may extend some amount past heel 8 of shoe 2 to provide additional stability. As users rarely want to be balanced on the backs of their heels. By extending along substantially the entire length of shoe 2, outrigger 4 can be formed along to the shoe providing additional functional width to shoe 2, thereby spreading the force on the outrigger when it is moved to engage surface 10 through the beginning of a foot roll, to provide the user increased stability.

In some embodiments, outrigger 4 may be formed integrally with the sole or a component of the sole of shoe 2, providing durability of outrigger 4. In some embodiments, an external or lower portion of outrigger 4 may be formed of the same material as the sole of shoe 2 with an additional core or filler for outrigger 4 having different elastomeric properties than the sole material. In such embodiments, the amount of lateral stiffness and comfort may be adjusted as desired. In other embodiments, outrigger may be formed integrally with another portion of the shoe above the sole, for example, in a cushion layer common to athletic shoes to which the sole material is adhered. The outrigger may molded from materials used as soles in shoes, such as plastic, rubber, rubber compounds, Vibram® or elastomer material. The outrigger may be formed entirely as a unitary member with the shoe sole, or formed as a member that is attached to the vamp or to the outer sole of the shoe. The vamp may also be formed to provide or support a portion of the outrigger.

The outrigger may extend laterally from the sole of the shoe, and is preferred to extend from the outer sole, and laterally to the side of the sole of the shoe. In some embodiments, the outrigger may not extend materially above a line defined by where the vamp meets the sole of the athletic shoe, it being recognized that athletic shoes generally have thicker soles than “street” shoes or dress shoes. In one embodiment, the outrigger may maintain a low profile, rising below the vamp of the shoe as the position of the talus within the shoe.

In some embodiments, outrigger 4 may be mounted along a lesser length of the shoe, such as from the ball, or widest part of the shoe to a point before middle of the heel. Such shorter outrigger designs may provide some roll protection while allowing for greater latitude in shoe design and style. However, the reduced length of the device may in some applications provide less roll protection than the longer lengths extending substantially the entire length of the shoe.

In one exemplary embodiment, outrigger 4 (e.g., a stabilizer member) may extend from the lateral side of the sole of the shoe from between about 1 to 2 cm from a normal width extent of a comparably sized conventional shoe. Outrigger 4 may be formed such that it extends from the most outwardly extending side of the shoe, whether the most outwardly extending portion is the vamp or the quarter of the shoe. Similarly, the outrigger may have different widths or provide variable width enhancements over a traditional width in varying amounts extending along the length of the shoe. This dimension, or additional width, may be provided sufficient to prevent inversion of the ankle of the user as the foot begins to roll under the user. Additional width beyond about 2 cm may interfere with activities of the user or with those in close contact with the user, but may be appropriate depending on the activity and the level of stability desired.

In other embodiments, such as is shown in FIG. 3, the outrigger may be a separate piece extending into and affixed to the sole of the shoe. Platform 14 may extend from within a portion of the sole and beyond the sole to the lateral side of the shoe, with additional outrigger material being mounted on and over the platform to provide stiffness and roll protection. The outrigger may be formed by a tube that is wrapped about its circumference in the material that is attached to the sole. In such an embodiment, the bottom surface of the outrigger may extend from the lateral side of the sole at an angle of 15° to 30°.

In another embodiment, the outrigger may have an arcuate bottom surface that curves out and away from the sole of the shoe, contacting the surface near the sole but arcing upwardly and above the surface as the bottom of the outrigger extends away from the sole. In another embodiment, the entire bottom surface of the outrigger may contact the surface.

The front of the outrigger may be formed in an arcuate shape as shown in FIG. 3, blending into the curve of the shoe. The arcuate shape is of such embodiments may generally be an extension of the arcuate front part of the outsole of most shoes, following the form of the shoe for an aesthetically pleasing look, while also reducing interference with normal use and activity while wearing the shoe.

A stabilizing member 110a-d, 115a-d, stabilizer, or outrigger may, for example, be incorporated into a slip-on member (e.g., an overshoe, a partial overshoe, etc.). Alternatively, or additionally, a stabilizing member 110a-d, 115a-d, stabilizer, or outrigger may, for example, be configured as a shoe insert. As yet another alternative, or addition, a stabilizing member 110a-d, 115a-d, stabilizer, or outrigger may, for example, include at least one adjustable strap (e.g., an ankle strap of a high-top shoe extending from a shoe sole to a shoe top, an ankle strap of a high-top shoe extending circumferentially around at least a portion of a person's ankle, a strap extending along shoe laces from a toe of the shoe to a top of the shoe, a strap incorporated into a shoe tong, etc.).

Any given stabilizing member 110a-d, 115a-d stabilizer, or outrigger may include an actuator (e.g., a pneumatic actuator, a mechanical actuator, a hydraulic actuator, an electrical actuator, etc.) that may be configured to vary a shape and/or orientation of the stabilizing member 110a-d, 115a-d, stabilizer, or outrigger. The shape and/or orientation of the stabilizing member 110a-d, 115a-d, stabilizer, or outrigger may be manually and/or automatically varied. For example, at least one sensor (e.g., a pressure sensor, a gyroscopic sensor, a level sensor, a motion sensor, strain sensor, etc.) may be incorporated within a shoe and connected to an actuator to automatically vary a shape and/or orientation of a stabilizing member 110a-d, 115a-d, stabilizer, or outrigger. Thereby, a user's stability and/or walking may be improved.

Output(s) of at least one sensor may be connected to an input(s) of a circuit (e.g., a discrete component electrical circuit, a processor, an application specific integrated circuit (ASIC), a field programmable gate array (FPGA), a logic circuit, etc.). Output(s) of the circuit may be connected to at least one actuator. The circuit may control at least one circuit output to automatically vary a shape and/or orientation of the stabilizing member 110a-d, 115a-d, stabilizer, or outrigger based on at least one circuit input. For example, an output of a sensor may indicate that a user has begun to lean in a certain direction (e.g., pressure on a sensor has increased/decreased, strain on a sensor has increased/decreased, etc.). The circuit may automatically vary a shape and/or orientation of the stabilizing member 110a-d, 115a-d to at least partially compensate (or correct) the lean of the person (i.e., the circuit may automatically vary a shape and/or orientation of the stabilizing member 110a-d, 115a-d, stabilizer, or outrigger via at least one actuator).

With reference to FIGS. 4A and 4B, a pair of therapeutic shoes 400a,b may include a right foot shoe 405ar,br and a left foot shoe 405al,bl. The therapeutic shoes 400a, b may include a plurality of stabilizing members 410b slidingly engaged within respective receptacles 412a. The stabilizing members 410b may be configured to be slid from side to side of a respective shoe to, for example, lengthen or shorten an amount that the respective stabilizing members 410b extends from a side of the shoe.

Turning to FIG. 5, a pair of therapeutic shoes 500 may include a right foot shoe 505r and a left foot shoe 5051 having at least three stabilizing sections 525, 530, 535 and at least two manual actuators 526, 536 (e.g., a pneumatic pump, a hydraulic pump, a mechanical device, etc.).

With reference to FIGS. 6A-6D, a system for configuring a therapeutic shoe 600a-d may include a left shoe and/or a right shoe 605b having a plurality of sensors 675a communicatively connected to a therapeutic shoe controller 680a, a plurality of inwardly extending stabilizing members 610a,b removable attached via inwardly extending stabilizing member attachments 612b and inwardly extending stabilizing member fasteners 611b, and a plurality of outwardly extending stabilizing members 615a,b removable attached via outwardly extending stabilizing member attachments 617b and outwardly extending stabilizing member fasteners 616b. Each inwardly extending stabilizing member 610a,b and each outwardly extending stabilizing member 615a,b may include at least one sensor and/or actuator 670a communicatively connected to the therapeutic shoe controller 680a.

The therapeutic shoe controller 680a may include a memory 681a storing a module 682a, a processor 683a, and a transceiver 684a. The processor 683a may execute the module 682a to, for example, cause the processor 683a to generate a pedograph 692. As illustrated in FIG. 6A, the pedograph 692 may, for example, indicate that a shoe wearer has an unsupported high arched foot. Additionally, or alternatively, a pedograph may indicate excessive pressure on the metatarsal heads (e.g., under a second metatarsal head). Because the arch is not being supported, a mid-foot pressure may be, for example, exerted on a lateral column of the associated foot (opposite of the arch). A pedograph may, for example, indicate more pressure on an outside part of a heel as well. A therapeutic shoe configuration may be determined, based on pedograph data, that includes at least one stabilizing member on a lateral (outside) aspect of the shoe.

The system for configuring a therapeutic shoe 600a-d may also include a remote computing device 685a communicatively connected to the therapeutic shoe controller 680a via a remote transceiver 689a, a communication network 690a (e.g., wired or wireless), and the shoe controller transceiver 684a. While not shown in FIGS. 6A-D, the system for configuring a therapeutic shoe 600a-d may include a second therapeutic shoe controller 684a within a second shoe. For example, a right therapeutic shoe may include a first therapeutic shoe controller 684a and a left therapeutic shoe may include a second therapeutic shoe controller 684a. The first therapeutic shoe controller 684a and the second therapeutic shoe controller 684a may communicate sensor and/or actuator data to/from one another. Thereby, the right therapeutic shoe may be dynamically controlled based on data from a left therapeutic shoe, and vice versa.

The remote computing device 685a may also include a memory 686a storing a module 687a, a processor 688a, a user input device 691, and a display device 695.

With further reference to FIG. 6C, a therapeutic shoe controller 680c may include a memory 681c. The therapeutic shoe controller 680c may be similar to, for example, the therapeutic shoe controller 680a and/or the remote computing device 685a of FIG. 6A. The memory 681c may be similar to, for example, the memory 681a and/or the memory 686a. The therapeutic shoe controller 680c may include a user interface generation module 610c, a shoe wearer personal information data receiving module 615c, an empirical shoe wearer/shoe configuration correlated data receiving module 620c, a right foot pedograph data receiving module 625c, a left foot pedograph data receiving module 630c, a right shoe configuration data generation module 635c, a left shoe configuration data generation module 640c, a shoe wearer and shoe configuration data storage module 645c, a shoe manufacture module 650c, a generic shoe modification module 655c, and a dynamic shoe configuration module 660c, stored on, for example, a set of computer-readable instructions. The modules 610c-660c may be similar to, for example, modules 682a and/or 687a of FIG. 6A. The modules 610c-660c may be stored on the memory 681c, as a set of computer-readable instructions, that, when executed by a processor (e.g., processor 683a, 688a of FIG. 6A) may cause the processor 683a, 688a to, for example, receive pedograph data and generate therapeutic shoe configuration data based on the pedograph data. Alternatively, some or all of the modules 610c-660c may be configured as hardware (e.g., electrical circuitry with discrete components, logic circuits, a field programmable gate array (FPGA), an application specific integrated circuit (ASIC), dedicated electrical circuits, etc.).

With further reference to FIG. 6D, a method of configuring a therapeutic show may be implemented by, for example, a processor (e.g., processor 683a of FIG. 6A) of a therapeutic shoe controller (e.g., a therapeutic shoe controller 680a of FIG. 6A) or a processor (e.g., processor 688a of FIG. 6A) of a remote computing device (e.g., a remote device 685a of FIG. 6A) each executing a portion of the modules 610c-660c of FIG. 6C.

In particular, the processor 683a may execute the user interface generation module 610c to, for example, cause the processor 683a to generate a user interface (block 610d). The user interface may be configured to, for example, allow a pedorithist, a podiatrist, a show wearer, etc. to enter pedograph data and/or personal information data.

The processor 683a may execute the shoe wearer personal information data receiving module 615c to, for example, cause the processor 683a to receive personal information from a shoe wearer (block 615d). The personal information data may be, for example, representative of at least one of: a shoe wearer's age; gender; height; weight; whether the shoe wearer has a previously configured pair of therapeutic shoes and, of so, associated configuration data for the previously configured pair of therapeutic shoes; details related to any shoe wearer falls or near falls; etc.

The processor 683a may execute the empirical shoe wearer/shoe configuration correlated data receiving module 620c to, for example, cause the processor 683a to receive empirical shoe wearing/shoe configuration correlated data (block 620d). The empirical shoe wearer/shoe configuration correlated data may be, for example, representative of previously configured therapeutic shoe configurations correlated with respective shoe wearer personal information data and/or respective pedograph data.

The processor 683a may execute the right foot pedograph data receiving module 625c to, for example, cause the processor 683a to receive right foot pedograph data (block 625d). The right foot pedograph data may be representative of, for example, pressures/pressure locations on a user's right foot when the user stands, walks and/or runs.

The processor 683a may execute the left foot pedograph data receiving module 630c to, for example, cause the processor 683a to receive left foot pedograph data (block 630d). The left foot pedograph data may be representative of, for example, pressures/pressure locations on a user's left foot when the user stands, walks and/or runs.

The processor 683a may execute the right shoe configuration data generation module 635c to, for example, cause the processor 683a to generate right shoe configuration data (block 635d). The right shoe configuration data may be representative of location(s) and/or length(s) of stabilizer member(s) on a right shoe.

The processor 683a may execute the left shoe configuration data generation module 640c to, for example, cause the processor 683a to generate left shoe configuration data (block 640d). The left shoe configuration data may be representative of location(s) and/or length(s) of stabilizer member(s) on a left shoe.

The processor 683a may execute the shoe wearer and shoe configuration data storage module 645c to, for example, cause the processor 683a to store shoe wearer and shoe configuration data (block 645d). The shoe wearer and shoe configuration data may be representative of shoe configuration data correlated with respective shoe wearer personal data and/or respective pedograph data.

The processor 683a may execute the shoe manufacture module 650c to, for example, cause the processor 683a to control an automatic shoe manufacturing module (650d). The processor 683a may execute the generic shoe modification module 655c to, for example, cause the processor 683a to implement a modification of a generic shoe (or level one stability shoe) (block 655d).

The processor 683a may execute the dynamic shoe configuration module 660c to, for example, cause the processor 683a to dynamically configure at least one therapeutic shoe (block 660d). A therapeutic shoe may, for example, be automatically and dynamically reconfigured while a shoe wearer is standing and/or moving. The dynamic shoe configuration may be, for example, based on real-time sensor data.

The processor 683a may execute a pedograph data receiving module to receive pedograph data. The pedograph data may be representative of a distribution of pressure resulting from at least one foot of an individual on a surface. The processor 683a may execute a therapeutic shoe configuration data generation module to generate therapeutic shoe configuration data based on the pedograph data. The pedograph data may be representative of at least one of: an output from at least one pressure sensor, an output of at least one strain sensor, an output of at least one digital image sensor, or an output from at least one pedograph. Alternatively, or additionally, the pedograph data may be representative of a distribution of pressure resulting from a left foot of the individual and the therapeutic shoe configuration data is representative of a configuration of at least one stabilizer member of a therapeutic shoe for a right foot. Alternatively, or additionally, the pedograph data may be representative of a distribution of pressure resulting from a right foot of the individual and the therapeutic shoe configuration data may be representative of a configuration of at least one stabilizer member of a therapeutic shoe for a left foot. The therapeutic shoe configuration data generation module may include a probability function and the therapeutic shoe configuration data is representative of a plurality of probable therapeutic shoe configurations. The therapeutic shoe configuration data generation module may include a machine learning algorithm and the therapeutic shoe configuration data is based on empirical data of a plurality of therapeutic shoe configurations correlated with respective pedograph data.

The processor 683a may receive pedograph data, receive therapeutic shoe pedograph review data from a therapeutic shoe manufacturer, receive therapeutic shoe manufacturer determination of best therapeutic shoe configuration for stability, and receive a configured therapeutic shoe from the manufacture.

A method of configuring a therapeutic shoe 600d may include receiving pedograph data representative of at least one of: a footprint of a therapeutic shoe wearer, a previously determined configuration of a therapeutic shoe, or personal data associated with a therapeutic shoe wearer. The method may also include generating therapeutic shoe configuration data based on the pedograph data, and configuring a therapeutic shoe based on the therapeutic shoe configuration data. The pedograph data may be at least partially based on information received from a therapeutic shoe wearer in response to information received from the therapeutic shoe wearer in response to a questioner. The pedograph data may be at least partially based on information received from a therapeutic shoe wearer in consultation with a podiatrist. The pedograph data may be at least partially based on information received from a therapeutic shoe wearer in consultation with a therapeutic shoe manufacture. The pedograph data may be representative of a right foot of a therapeutic shoe wearer, and therapeutic shoe configuration data may be representative of a configuration of a left foot therapeutic shoe. The pedograph data may be representative of a left foot of a therapeutic shoe wearer, and the therapeutic shoe configuration data is representative of a configuration of a right foot therapeutic shoe. The therapeutic shoe configuration data may be representative of a level one stability therapeutic shoe, and the method may further include receiving level two stability data, and generating level two therapeutic shoe stability data based on the level two stability data.

A pair of therapeutic shoes may include a left shoe having at least one left shoe sensor and at least one left shoe stabilizer member, and a right shoe having at least one right shoe sensor and at least one right shoe stabilizer member. At least one left shoe stabilizing member may be dynamically configured based on an output from the at least one right shoe sensor or the at least one right shoe stabilizing member may be dynamically configured based on an output of the at least one left shoe sensor. A configuration of the at least one left shoe stabilizer member or the at least one right shoe stabilizer member may be automatically reconfigured in response to a change in the output of the at least one right shoe sensor or the output of the at least one left shoe sensor. The at least one left shoe stabilizer member and/or the at least one right shoe stabilizer member may include at least one actuator selected from: a pneumatic actuator, an electric actuator, a hydraulic actuator, or a mechanical spring actuator. The pair of therapeutic shoes may include a left shoe transceiver and a right shoe transceiver. The output of the at least one right shoe sensor may be received at the left shoe transceiver and/or an output of the at least one left shoe sensor may be received at the right shoe transceiver. At least one left shoe stabilizer may be removably secured to the left shoe and/or the at least one right shoe stabilizer may be removably secured to the right shoe. At least one left shoe stabilizer may be removably secured to the left shoe via at least one left shoe fastener and/or the at least one right shoe stabilizer may be removably secured to the right shoe via at least one right shoe fastener. At least one left shoe fastener and/or at least one right shoe fastener may be selected from: a mechanical fastener, a magnetic fastener, a pressure sensitive adhesive, an interference-fit assembly, or a friction-fit assembly.

This detailed description is to be construed as exemplary only and does not describe every possible embodiment, as describing every possible embodiment would be impractical, if not impossible. One could implement numerous alternate embodiments, using either current technology or technology developed after the filing date of this application.

What is claimed is:

1. A pair of therapeutic shoes, comprising:
a left shoe having at least one left shoe sensor and at least one left shoe stabilizer member; and
a right shoe having at least one right shoe sensor and at least one right shoe stabilizer member, wherein the at least one left shoe stabilizing member is dynamically configured based on an output from the at least one right shoe sensor or the at least one right shoe stabilizing member is dynamically configured based on an output of the at least one left shoe sensor.

2. The pair of therapeutic shoes of claim 1, wherein a configuration of the at least one left shoe stabilizer member or the at least one right shoe stabilizer member is automatically reconfigured in response to a change in the output of the at least one right shoe sensor or the output of the at least one left shoe sensor.

3. The pair of therapeutic shoes of claim 1, wherein the at least one left shoe stabilizer member and/or the at least one right shoe stabilizer member includes at least one actuator selected from: a pneumatic actuator, an electric actuator, a hydraulic actuator, or a mechanical spring actuator.

4. The pair of therapeutic shoes of claim 1, further comprising:
a left shoe transceiver and a right shoe transceiver, wherein the output of the at least one right shoe sensor is received at the left shoe transceiver and/or an output of the at least one left shoe sensor is received at the right shoe transceiver.

5. The pair of therapeutic shoes of claim 1, wherein the at least one left shoe stabilizer is removably secured to the left shoe and/or the at least one right shoe stabilizer is removably secured to the right shoe.

6. The pair of therapeutic shoes of claim 1, wherein the at least one left shoe stabilizer is removably secured to the left shoe via at least one left shoe fastener and/or the at least one right shoe stabilizer is removably secured to the right shoe via at least one right shoe fastener.

7. The pair of therapeutic shoes of claim 6, wherein the at least one left shoe fastener and/or the at least one right shoe fastener is selected from: a mechanical fastener, a magnetic fastener, a pressure sensitive adhesive, an interference-fit assembly, or a friction-fit assembly.

8. A computer-readable medium storing computer-readable instructions that, when executed by a processor, cause the processor to generate a configuration for at least one therapeutic shoe, the computer-readable medium comprising:
a pedograph data receiving module that, when executed by the processor, causes the processor to receive pedograph data, wherein the pedograph data is representative of a distribution of pressure resulting from at least one foot of an individual on a surface; and
a therapeutic shoe configuration data generation module that, when executed by the processor, causes the processor to generate therapeutic shoe configuration data based on the pedograph data.

9. The computer-readable medium of claim 8, wherein the pedograph data is representative of at least one of: an output from at least one pressure sensor, an output of at least one strain sensor, an output of at least one digital image sensor, or an output from at least one pedograph.

10. The computer-readable medium of claim 8, wherein the pedograph data is representative of a distribution of pressure resulting from a left foot of the individual and the therapeutic shoe configuration data is representative of a configuration of at least one stabilizer member of a therapeutic shoe for a right foot.

11. The computer-readable medium of claim 8, wherein the pedograph data is representative of a distribution of pressure resulting from a right foot of the individual and the therapeutic shoe configuration data is representative of a configuration of at least one stabilizer member of a therapeutic shoe for a left foot.

12. The computer-readable medium of claim 8, wherein the therapeutic shoe configuration data generation module includes a probability function and the therapeutic shoe configuration data is representative of a plurality of probable therapeutic shoe configurations.

13. The computer-readable medium of claim 8, wherein the therapeutic shoe configuration data generation module includes a machine learning algorithm and the therapeutic shoe configuration data is based on empirical data of a plurality of therapeutic shoe configurations correlated with respective pedograph data.

14. A method of configuring a therapeutic shoe, the method comprising:
receiving pedograph data representative of at least one of: a footprint of a therapeutic shoe wearer, a previously determined configuration of a therapeutic shoe, or personal data associated with a therapeutic shoe wearer;
generating therapeutic shoe configuration data based on the pedograph data; and
configuring a therapeutic shoe based on the therapeutic shoe configuration data.

15. The method of claim 14, wherein the pedograph data is at least partially based on information received from a therapeutic shoe wearer in response to information received from the therapeutic shoe wearer in response to a questioner.

16. The method of claim 14, wherein the pedograph data is at least partially based on information received from a therapeutic shoe wearer in consultation with a podiatrist.

17. The method of claim 14, wherein the pedograph data is at least partially based on information received from a therapeutic shoe wearer in consultation with a therapeutic shoe manufacture.

18. The method of claim 14, wherein the pedograph data is representative of a right foot of a therapeutic shoe wearer, and wherein the therapeutic shoe configuration data is representative of a configuration of a left foot therapeutic shoe.

19. The method of claim 14, wherein the pedograph data is representative of a left foot of a therapeutic shoe wearer, and wherein the therapeutic shoe configuration data is representative of a configuration of a right foot therapeutic shoe.

20. The method of claim 14, wherein the therapeutic shoe configuration data is representative of a level one stability therapeutic shoe, and wherein the method further comprising:
receiving level two stability data; and
generating level two therapeutic shoe stability data based on the level two stability data.
